(19) United States
(12) Patent Application Publication
(10) Pub. No.: US 2026/0096730 A1
(43) Pub. Date: Apr. 9, 2026

(54) Mobile Rheometer Device and Method for Medical Diagnostics of Human, Animal or Plant Samples

(71) Applicant: NETZSCH-Gerätebau GmbH, Selb (DE)

(72) Inventors: Rebekka Taubmann, Selb (DE); Florian Rummel, Selb (DE); Sabrina Küspert, Selb (DE); Levente Szántó, Hof (DE)

(21) Appl. No.: 19/350,345

(22) Filed: Oct. 6, 2025

(30) Foreign Application Priority Data
Oct. 9, 2024 (DE) 10 2024 129 203.2

(51) Int. Cl. A61B 5/02; A61B 5/00; A61B 5/0265; A61L 9/18

(52) U.S. Cl. CPC A61B 5/02035; A61B 5/0265; A61B 5/7271; A61L 9/18; A61B 2503/40; A61B 2560/0219; A61L 2103/15

(57) ABSTRACT

A mobile rheometric device for medical diagnosis of human, animal, or plant samples. Such a mobile rheometric device thus includes a rheological measurement device, to which is connected an evaluation unit having an evaluation program as well as, connected thereto, an output unit. The evaluation unit with evaluation program is configured to classify the measured rheological parameters of a human, animal, or plant sample with respect to at least one medical diagnosis or into relevant parameter groups for a medical diagnosis, so that a detailed view of the sample can be provided to a user of the mobile rheometric device by means of the output unit. A corresponding method is also disclosed.

### TECHNICAL FIELD

The present invention relates to a mobile rheometric device as well as a method for medical diagnosis of human, animal, or plant samples.

### BACKGROUND

To be able to obtain far-reaching data concerning human, animal, or plant samples, such material is frequently analyzed at considerable expense in the laboratory after extraction from the organism. Alternatively, it is also customary to investigate a fluid that is related to the tissue or organism that is to be analyzed.

Thus, for instance, a sample is extracted from a human being during a biopsy or other procedure by medical specialists and is then turned over to pathologists. In the pathology work, a histological investigation often follows, typically with the staining of tissue and subsequent pathological diagnosis. Tissue samples can also be prepared by tissue sectioning for purposes of further analysis of these individual sections.

Rather than a histological examination with staining and the use of microscopic devices, rheological techniques may also be used, such as in order to perform a medical diagnosis. These techniques can include, for example, rotation rheometers or capillary rheometers. In addition, falling-sphere viscometers or microfluidic devices can be employed. Pathological tissue is distinguished from the physiological condition, for example, by its viscous, viscoelastic, or elastic properties.

For example, stenoses or similar disorders can be investigated more closely by such means. Bodily fluids such as saliva, blood, and the like are likewise distinguished in their viscous, viscoelastic, or elastic properties when a modification occurs. Such a situation is known, for example, in association with sickle cell anemia. In such cases, knowledge of rheological characteristics of physiological or pathological properties can be put to diagnostic use.

In this process, measurements of one or more rheological values, such as (sheer) viscosity, extensional viscosity, shear stress, viscoelastic properties, storage modulus, or loss modulus can be taken. Such measurements can include absolute or relative values. A particular sample can typically be set into oscillation or vibration. Alternatively, rotation or motion in the form of a flow can also be obtained. In addition, bodily determined circumstances of samples can already assume forms of oscillation, vibration, rotation or flowing, and thus this circumstance can be useful for rheological methods.

Procedures of this kind employing various applications of such techniques can entail a certain time commitment. In addition, in transporting samples a certain logistical infrastructure is required in order to avoid impairments to samples which are often sensitive during this time. In this context, modifications are probable despite all precautions and cannot be entirely ruled out. Biological sample material is generally modified fairly rapidly, and thus subsequent investigations and/or analysis is adversely affected. In the extraction of samples or during transport of samples, particularly biological samples, there is also a risk, particularly with biological samples, of unintentional infection when such contact with these samples occurs during the aforementioned activities.

In the aforementioned cases of biopsy and extraction during surgery, the necessary medical infrastructure is often available only with maximal providers. Supply and diagnosis in remote locations, such as at high elevations or at sea, can accordingly prove difficult, since the necessary medical infrastructure is either only available with difficulty or not present at all.

### SUMMARY

Given this background, the object of the present invention is to provide a mobile rheometric device and a method which can overcome the aforementioned disadvantages, at least in part.

This object is fulfilled by means of a mobile rheometric device having the characteristics of patent claim 1 as well as a method having the characteristics of patent claim 10.

A mobile rheometric device is accordingly foreseen for medical diagnosis of human, animal, or plant samples. Such a mobile rheometric device in this case includes a rheological measurement device, an evaluation unit connected with the former, having an evaluation program, and connected therewith, an output unit. Thus the evaluation unit is configured with an evaluation program in order to classify the measured rheological parameters of a human, animal, or plant sample with respect to at least one medical diagnosis or into relevant parameter groups for a medical diagnosis, so that a precise view of the sample can be provided to the user of the mobile rheometric device by means of the output unit.

In addition, a method is foreseen for the medical diagnosis of human, animal, or plant samples. Such a method comprises the following steps: provision and activation of an inventive mobile rheometric device; measurement of a human, animal, or plant sample by means of the mobile rheometric device; classification of results of the rheological measurements with respect to at least one medical diagnosis by means of the mobile rheometric device; outputting the classified results by means of the mobile rheometric device so that a detailed view of the sample can be provided to a user of the mobile rheometric device by means of the mobile rheometric device.

One idea on which the invention is based consists in providing a mobile technical solution, which can be employed directly and locally in such a way that there is direct access to the measurement results of samples that are to be analyzed. The inventive mobile rheometric device offers the advantage that it can easily be moved inside the respective application site, wherein on the basis of the specially foreseen evaluation unit it is possible to perform a classification of measurement values in the immediate location. Thus any costly transport and complex evaluation methods can be avoided. In particular, samples subject to rapid modification over time, and which thus can sometimes become unusable for a desired analysis, can thus be analyzed better and more consistently.

By dispensing with a necessarily complex infrastructure of a maximal provider, it becomes possible to make a pathological and/or generally medical diagnosis accessible to a considerable number of people. Persons whose course of illness must be tracked can presumably do so at home and, if necessary, remotely, so that they are obliged to make contact with medical specialists less frequently or only when needed. The foreseen mobile rheometric device can also be used advantageously on a broader basis in connection with the earliest possible diagnoses and thus with an improved outlook concerning a recovery process or in relation to prevention more generally.

To this extent, the rheometric device and the corresponding method can be used advantageously as part of the equipment of operating rooms and medical practices as well as for use with mobile medical provider installations or units. Even certain individual uses may be possible by medical lay persons, such as directly by a patient, while the mobile rheometric device can be used advantageously, similarly to a personal appliance, as a mobile solution for medical diagnostic activities or the like.

One embodiment of the mobile rheometric device foresees that the evaluation unit including its evaluation program, for the purpose of classification, can be combined with at least one database for medical diagnosis of human, animal, or plant samples based on rheological parameters, or that it at least includes such a database.

In this manner, a particularly rapid and far-reaching classification can be performed. Also, depending on the database employed, the range of application of the inventive mobile rheometric device can be extended. In addition, it thus becomes more probable that, for application in remote areas, the use of the inventive mobile rheometric device can be considered sufficient for a consistent analysis.

Thus the rheological properties can likely be coordinated with the database (or with more than one database) containing information on the rheological properties of a physiological or pathological nature. By means of this coordination, the rheometric device makes it possible to describe one or more indications for the diagnosis (or for the professional medical user) in relation to the condition of the sample. Indications of a general nature, in the form of parameters or the like, can also be derived, which are also easily understandable and accessible for a medical lay person.

By means of the inventive mobile rheometric device and the corresponding method, it thus becomes possible to conduct rheological examinations with reference to a medical diagnosis of human, animal, or plant samples in the immediate spatial and temporal proximity. The rheological examinations here allow certain measurements under the most physiological conditions possible, for instance with respect to a pH value or to a certain temperature, an ionic concentration, concerning available enzymes or other parameters, such as for bodily fluids.

According to a refinement of the mobile rheometric device, it is foreseen that the evaluation program can be adaptable, by means of at least one user-defined input, so that an individualized use of the respective database is possible.

Thus it becomes possible to conduct an even more specialized evaluation routine in the immediate location. The evaluation program here can be adapted with the simplest inputs, so that an even better use of the connected databases can be possible by means of the inventive mobile rheometric device. For example, it can hereby become possible to correct influences of factors in the rheological measurement that are not necessarily or exclusively associated with a possible diagnostic illness, so that thereafter it is possible to draw some comparison with information from the database and/or databases. In this context another possibility would be current algorithms as an optional part of the evaluation program, on the basis of artificial intelligence, or also algorithms as an optional part of the evaluation, on the basis of current machine programs. It can be foreseen that the evaluation program is configured to process a number of inputs in such a way so that a clear allocation can be assured in the sense of the present invention, that is, a most unequivocal possible classification with respect to a diagnosis and/or the parameters relevant for a diagnosis can be assured.

Another refinement of the mobile rheometric device foresees that the at least one user-defined input is selected from: gender-sensitive inputs, nutritional input in human or animal subject groups, medication ingestion by a human or animal subject, fertilizer application on plant subjects, plant protection application on plant subjects, at least one indication on correlated influences of medications and pathological modifications in at least one organism, suspected diagnoses; at least one indication from a patient register from at least one country, at least one indication on a prior illness of the organism being examined.

These diverse inputs can thus be employed to configure even better the classifications and the comparisons they employ with the database or databases. In particular, gender-sensitive inputs can advantageously be used to configure more precise statements and/or classifications, since thereby the consideration of gender-specific differences can be directly integrated in the evaluation process.

The at least one indication on correlated influences of medications and pathological modifications in at least one organism can be present, in particular, as a recognition such as has occurred in India between the use of diclofenac in livestock and the death of vultures. Because diclofenac was administered to livestock, this drug was absorbed into the carcass and thus into the various organisms of the local vulture population, leading to liquidation of vultures, for which the medication was fatal.

One embodiment of the mobile rheometric device foresees that the rheological measurement device is configured to measure rheological parameters of the human, animal, or plant samples separately or on the organism.

This results in an especially flexibly usable mobile rheometric device. The device can either be applied directly on the organism that is to be sampled, without causing it any notable impairment. This offers the advantage that the sample, that is, the area of the organism to be investigated need not be disturbed but instead, after the examination, is left essentially in its original condition. In particular, this result is favorable in tumor-type modifications of tissue sections in a human or animal body, to avoid introducing modified cell tissue unintentionally into the circulation system of the remaining organism. In other words, the inventive mobile rheometric device also can be used in or on tissue, for example before the actual extraction of the tissue or generally of the material sample. Thus the tissue remains intact and is only surgically extracted when this circumstance proves necessary because of the completed medical diagnosis. Analogously, then, for a plant organism, we can determine which parts are to be extracted, and in what quantity. For example, depending on a mushroom or pest infestation.

Alternatively, the device can be placed in the immediate vicinity of the organism that is to be sampled, so that extracted samples can be examined immediately. For instance, take the case of a tissue sample of a human or animal body, which is extracted during a biopsy or a surgical procedure. This could also be a extracted portion of a plant body and/or plant organism.

An embodiment of the mobile rheometric device foresees that the mobile rheometric device includes an energy system, which is configured to supply the mobile rheometric device with electrical energy autarchically or by means of an external energy supply network.

The energy system can possibly include rechargeable accumulator systems or the like, so that both a battery-operated mode as well as a network mode is possible. The mobile rheometric device can advantageously be used even more flexibly as a result.

According to a refinement of the inventive device, the mobile rheometric device can essentially comprise external materials, in particular stainless steel or titanium, which are effective for a sterilization method, in particular gamma ray sterilization, so that the mobile rheometric device can be used for sterilization of medicinal products after completion of a sterilization procedure in an operating room or even during production or with a service provider.

To this extent, the mobile rheometric device can be hygienically designed and/or configured and thus can advantageously be used in a rigorously sterile environment, such as an operating room or the like. In this connection, external materials in particular are the materials and sites of the device that essentially shield the interior main components of the device from the outside world or at least partly enclose it. Complete enclosure can also be provided.

One embodiment of the mobile rheometric device foresees that the mobile rheometric device is configured so as simultaneously to measure and classify a number of samples.

In this manner the mobile rheometric device can be used even more efficiently, so that costs per examination sample can be reduced.

Another embodiment of the mobile rheometric device foresees that the mobile rheometric device is configured to be controlled by means of at least one external appliance that can be coupled with the mobile rheometric device. Such an external device can be, for instance, a smart phone, a tablet or a similar mobile terminal.

For these purposes the evaluation program, for example, can be expanded to comprise a shape that can be installed on the external appliance in the form of an application or the like. Therefore the evaluation program, in other words, can be configured to provide this application and, for instance, by downloading or the like, to transfer it to the external appliance so that the mobile rheometric device can be controlled by means of the external appliance, which can be coupled. It is also possible in this context that the evaluation program is configured to provide this function in connection or by means of a corresponding cloud support. The external appliance can also be foreseen, at least partly, as a cloud-based technical solution.

An embodiment of this method provides that the following additional steps in the method are foreseen: connection of the evaluation unit and evaluation program of the mobile rheometric device with at least one database for medical diagnosis of human, animal, or plant samples on the basis of rheological parameters; adjustment of the evaluation program by mean of at least one user-defined input, so that an individualized use of the connected external database is possible.

The aforementioned advantages are also valid, to the extent transferrable, for this variant of the inventive method. In particular, the inventive method can be used advantageously, in principle, to make possible a gender-sensitive evaluation so that a support of a medicinal evaluation that takes gender-specific differences into account becomes possible.

### BRIEF DESCRIPTION OF THE DRAWINGS

The invention is hereinafter clarified with reference to the drawings, which are as follows:

FIG. 1 shows a schematic depiction of a mobile rheometric device according to an embodiment of the present invention.

FIG. 2 shows a schematic depiction of a user scenario of a mobile rheometric device according to an embodiment of the present invention.

FIG. 3 shows a schematic depiction of an additional user scenario of a mobile rheometric device according to an embodiment of the present invention.

FIG. 4 shows a schematic flow diagram for a method according to an embodiment of the present invention.

FIG. 5 shows a schematic depiction of an additional user scenario of a mobile rheometric device according to an embodiment of the present invention.

FIG. 6 shows a schematic depiction of an additional user scenario of a mobile rheometric device according to an embodiment of the present invention.

FIG. 7 shows a schematic depiction of an additional user scenario of a mobile rheometric device according to an embodiment of the present invention.

The same reference numbers in the drawings designate the same or functionally identical components, unless otherwise indicated.

### DETAILED DESCRIPTION

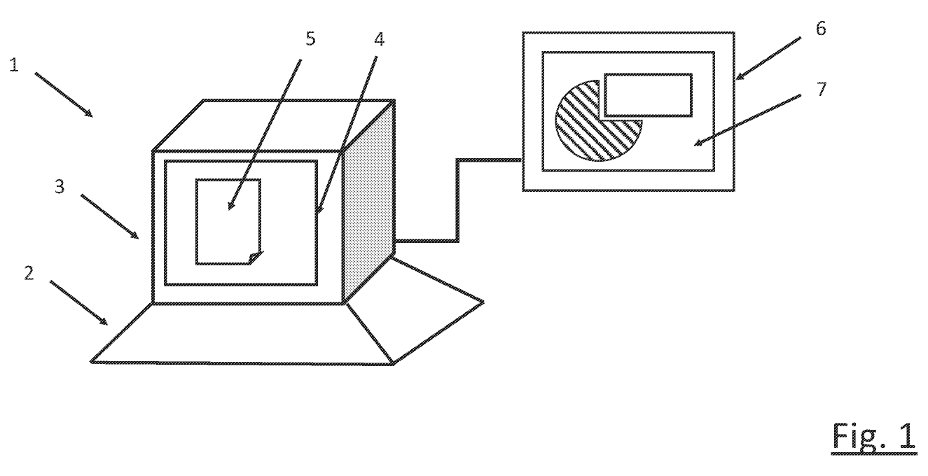

FIG. 1 shows a schematic depiction of a mobile rheometric device 1 according to an embodiment of the present invention. In particular, it relates to a mobile rheometric device 1 for medical diagnosis of human, animal, or plant samples. The samples can consist, for example, of human tissue samples.

The mobile rheometric device 1 here is depicted in markedly simplified form with a rheological measurement device 2, which, in relation to the image plane, is situated below a main body 3 of the mobile rheometric device 1. In variants of embodiments (not shown in detail) of the inventive mobile rheometric device 1, it is conceivable for the rheological measurement device 2 to be situated at least partly or even entirely inside the main body 3. Partially lateral arrangements or a complete lateral arrangement can be foreseen. It is also conceivable that the rheological measurement device 2 is provided separately and is coupled with the main body 3 and the components situated with it by means of a cable connection or wireless connection. In an additional embodiment, accordingly, it is also conceivable that the evaluation unit 4, with the evaluation program 5, is additionally (in the case of the presence of wireless connection techniques or the like) or generally contained in a mobile terminal or installed there or arranged there or generally retained there or, by means of a technical supplement, virtually or even physically foreseen and/or accordingly positioned there as a separate subunit of the mobile rheometric device 1.

An evaluation unit 4 with evaluation program 5 is depicted in the main body 3 of the mobile rheometric device 1. This evaluation unit 4 with evaluation program 5 is connected with the measurement device 2 by a connecting means not depicted further, so that any measurement values measured by means of the rheological measurement device 2 can be transmitted to the evaluation unit 4 and evaluation program 5.

The evaluation unit 4 with evaluation program 5 is thus configured to classify the measured rheological parameters of a human, animal, or plant sample with respect to at least one medical diagnosis, so that a detailed view of the sample can be provided to a user of the mobile rheometric device 1 by means of an output unit 6 that is illustrated and connected with the evaluation unit 4 and evaluation program 5.

The illustrated output unit 6 is thus essentially designed in the form of a monitor with a display screen 7 suitable for depicting classified results. This display screen 7 can be configured to present color images in two- or three-dimensional views, as is illustrated here in FIG. 1 schematically with a ¾-circular symbol and a box.

Here the ¾ circular symbol can represent healthy tissue while the box depicts conspicuous tissue on the basis of the classification. The output unit 6 is shown as coupled by a first connecting cable 8 with the main body 3 of the mobile rheometric device 1 and thus with the evaluation unit 4 and its evaluation program 5, so that a detailed view of the sample is provided to a user of the mobile rheometric device 1 by means of the output unit 6.

Optionally, the mobile rheometric device 1 can be configured to be used for a number of samples that are to be simultaneously evaluated, for instance in order to improve the statistical informative value of the examination.

The at least one database (not further depicted) can be coupled with the mobile rheometric device 1 by using any connecting means, for example cable-based or also by means of wireless technology. Also conceivably, the at least one database (not depicted further) can be part of the mobile rheometric device 1, at least to some extent. For example, the at least one database (not depicted further) can thus be, at least partially, a part of the evaluation unit 4 with its evaluation program 5. It is also conceivable that the at least one database (not further depicted) is a separate unit of the mobile rheometric device 1, wherein it is accordingly designed as coupled with the other components of the mobile rheometric device 1 and mounted inside it.

The at least one database (not presented in greater detail) can thus be either a fixed component of the mobile rheometric device 1 or can be linked with it by way of interfaces of the mobile rheometric device 1. In both cases, thus, the at least one database can enable medical specialist personnel, during or after the measurement, to directly make an evaluation of the rheological parameters and a diagnosis connected thereto.

The at least one database, as already mentioned, can be provided with additional inputs, such as additional data, for instance indications concerning nourishment or a medication of the patient, so that correlations and corrections can thus be made which, with these new inputs, allow an individualized form of the at least one database. In this manner, for instance, it is possible to correct the influence of factors that are not necessarily or exclusively associated with the diagnosed illness. In such case the mobile rheometric device 1 can make an evaluation with sensitivity to gender and thus support medical evaluation which takes gender-specific differences into account.

Specially configured dimensions for the mobile rheometric device 1, for instance one-way geometries, can be foreseen here, so that a standardized sample task is possible on the mobile rheometric device 1.

The mobile rheometric device 1, in an embodiment that is not depicted in detail, can have a mechanism at its disposal, for example in the form of software or the like in the form of an application, which standardizes the procedure of taking the sample, implementing the sample, measuring it and evaluating it, and in particular classifying it, to such an extent that interpersonal differences based on the operating staff or an individual operator can be minimized.

As already mentioned, the mobile rheometric device 1 can take a portable form. The mobile rheometric device 1 can rely either on network or on battery operation, thus enabling use both in stationary and mobile modes. For example, the mobile rheometric device 1 can thus be operated autarchically in remote locations. Accordingly, the inventive mobile rheometric device 1 is a portable medical device, which can serve in the most autonomous way even in more remote areas without highly developed infrastructure.

The mobile rheometric device 1 can be employed mounted on the body, so that the sample can be obtained without invasive intervention in the human, animal or plant body and thus is usable by medical laypersons. The focus during such a extraction and/or application can be on personal interaction, and in that process only mere indications can be communicated, that is, no diagnoses, which are reserved to medical specialists.

Structurally, the mobile rheometric device 1, particularly with regard to its spatial mass, can be a hand-held device. To this extent the mobile rheometric device 1 can take the form of a small appliance, which an operator can hold comfortably and without great effort in a single hand. Thus there is the further advantage that the inventive mobile rheometric device 1 is designed to allow light construction, so that mobile use is possible in addition to stationary application.

In addition, the mobile rheometric device 1 can be produced in an embodiment (not depicted in further detail) with a smart phone, tablet or similar mobile terminal or allowing control from a cloud source.

The mobile rheometric device 1 can be considered and/or designated as a special rheometer, which is configured for immediate analysis for medical specialists and medical laypersons in connection with a pathology database.

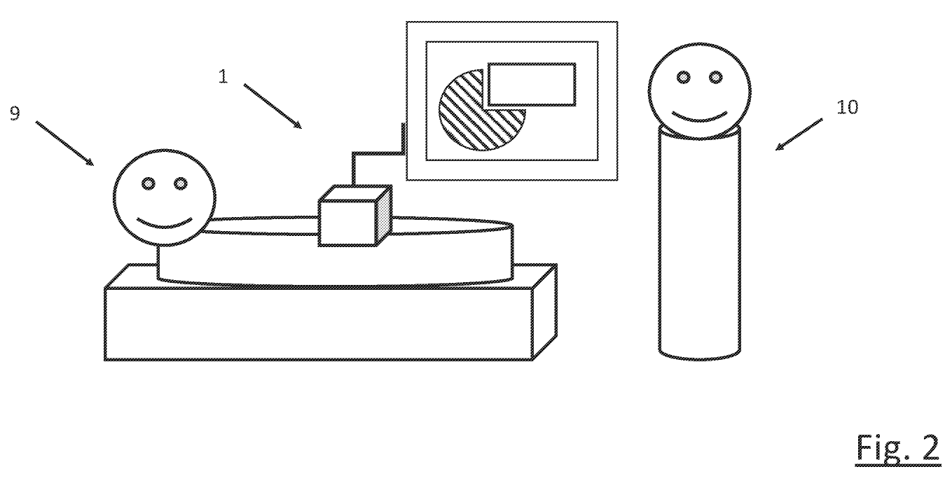

FIG. 2 shows a schematic depiction of a user scenario of a mobile rheometric device 1 according to an embodiment of the present invention. It can take the form, for instance, of a mobile rheometric device 1 according to FIG. 1.

The mobile rheometric device 1 here is shown in direct contact with a patient 9. It is conceivable, in this case, that the mobile rheometric device 1 and/or the related rheological measurement device 2 is foreseen at least partly as installed directly in the body of the patient 9 or only disposed on the body of the patient 9. In other words, the diagnosis can be conducted either directly in or on the body, whereas the mobile rheometric device 1 is configured with its corresponding components for both those variants.

In one variant, for example, it can be foreseen that the mobile rheometric device 1 is on the body of the patient 9 before extraction of a tissue sample, so that this sample accordingly can be diagnosed directly by the mobile rheometric device 1 when it is extracted.

A user 10 of the mobile rheometric device 1 is depicted to the right, on the basis of the image plane. This can be a medical staff person, for example an operator of the device.

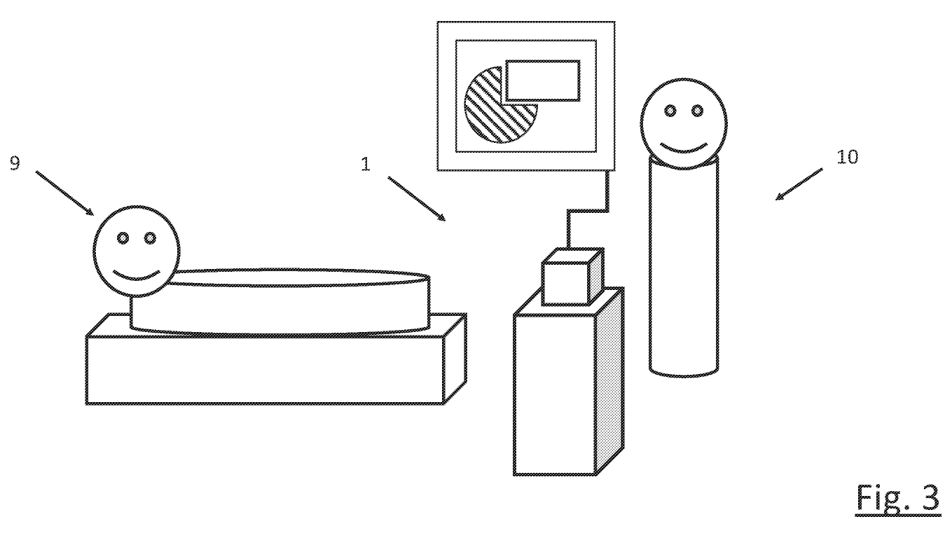

FIG. 3 shows a schematic depiction of an additional user scenario of a mobile rheometric device 1 according to an embodiment of the present invention. For example, this can be a mobile rheometric device 1 in accordance with FIG. 1.

The mobile rheometric device 1 in this case is not shown as directly in contact with a patient 9 but rather in the spatial proximity to the patient 9. For instance, the mobile rheometric device 1 is presented as being in the same operating room as the patient 9, so that the mobile rheometric device 1 is depicted during the procedure, or in spatial proximity to the body of the patient 9 that is to be examined.

The mobile rheometric device 1 is accordingly foreseen in spatial proximity, so that it can be put to use immediately after extraction of a sample, for example a tissue sample.

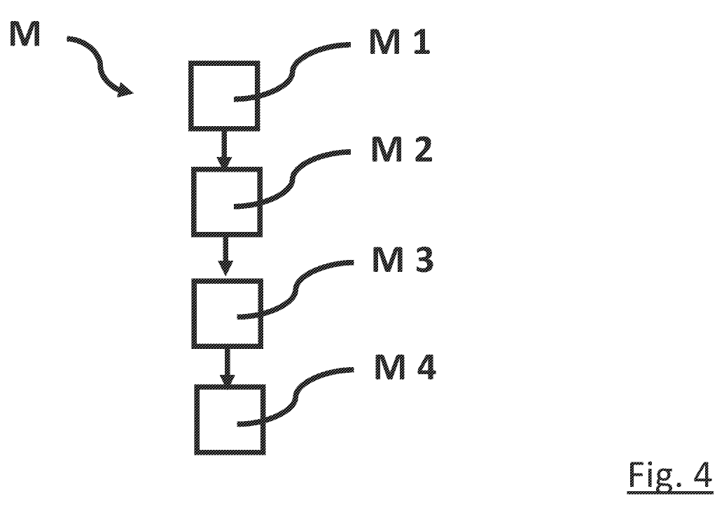

FIG. 4 shows a schematic flow diagram for a method M for the medical diagnosis of human, animal, or plant samples according to an embodiment of the present invention.

In a first step M1 of the method, an inventive mobile rheometric device is provided and activated. In a second step M2, human, animal, or plant samples are measured by means of the inventive mobile rheometric device.

In a third step M3 of the method, results of the rheological measurements are classified with respect to at least one medical diagnosis by means of the inventive mobile rheometric device. In a fourth step M4, the classified results are output by means of the inventive mobile rheometric device, so that a detailed view of the sample can be provided to a user of the mobile rheometric device by means of the mobile rheometric device.

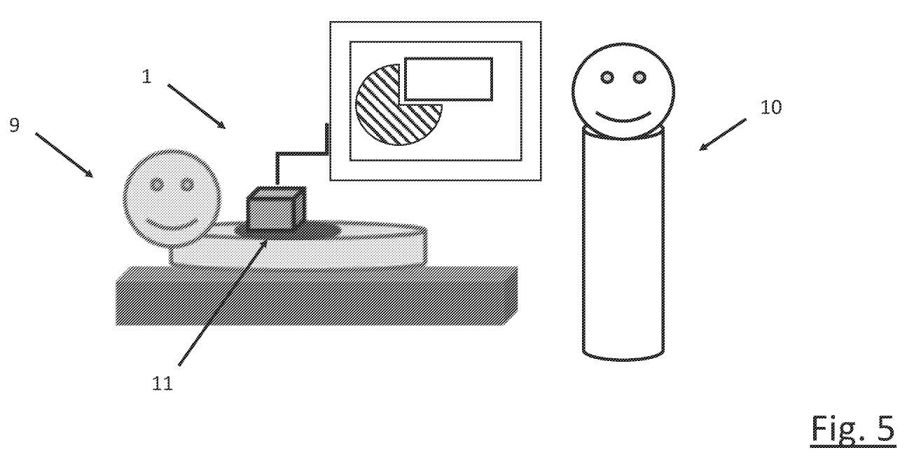

FIG. 5 shows a schematic depiction of an additional user scenario of a mobile rheometric device 1 according to an embodiment of the present invention. This can be, for example, a mobile rheometric device 1 in accordance with FIG. 1.

The mobile rheometric device 1 in this case is shown as in direct contact with a patient 9. It is conceivable here that the mobile rheometric device 1 and/or the related rheological measurement device 2 is to be placed at least partly directly inside the body of the patient 9 or else merely on the body of the patient 9. In other words, the diagnosis can be conducted either directly in or on the body, whereas the mobile rheometric device 1 with its corresponding components is configured for both variants.

Contrary to the depiction in FIG. 2, at the height of the abdomen of the patient 9 an opened site 11 of the body is depicted. In other words, in this case the body of the patient 9 is opened and subsequently the sample is characterized and/or measured on the opened site 11 of the body. This can be accomplished, for example, by mechanically contacting a firm bodily tissue, fluid, or digestive intermediate or end product or the like.

A user 10 of the mobile rheometric device 1 is depicted at the right on the image plane. This can be a medical staff person, for example an operator.

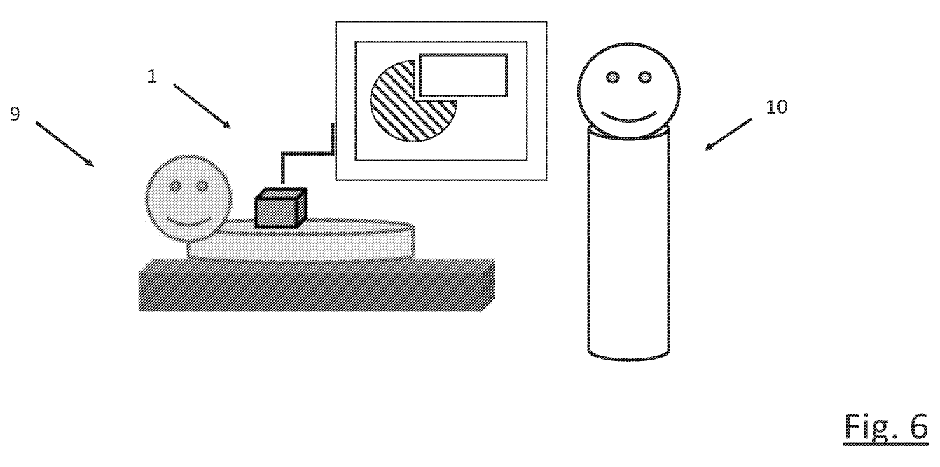

FIG. 6 shows a schematic depiction of an additional user scenario of a mobile rheometric device 1 according to an embodiment of the present invention. It can take the form, for instance, of a mobile rheometric device 1 according to FIG. 1.

The mobile rheometric device 1 here is shown in direct contact with a patient 9. It is conceivable in this case that the mobile rheometric device 1 and/or the related rheological measurement device 2 is presented at least partly directly inside the body of the patient 9 or only on the body of the patient 9. In other words, the diagnosis can either be performed directly in or on the body, so that the mobile rheometric device 1 with its corresponding components is configured for both variants.

Contrary to the depiction in FIG. 5, there is no open site shown at the abdominal level of the patient’s body.

The mobile rheometric device 1 and/or the related rheological measurement device 2 is positioned only on the surface of the body of the patient 9. This can be accomplished, for instance, with low normal force. In other words, the mobile rheometric device 1 and/or the related rheological measurement device 2 is held in place with low normal force. Thereafter, for example, an oscillating measurement of the skin tissue, for example, can be performed.

To the right on the image plane, a user 10 of the mobile rheometric device 1 is shown. This can be a medical staffer, such as an operating technician.

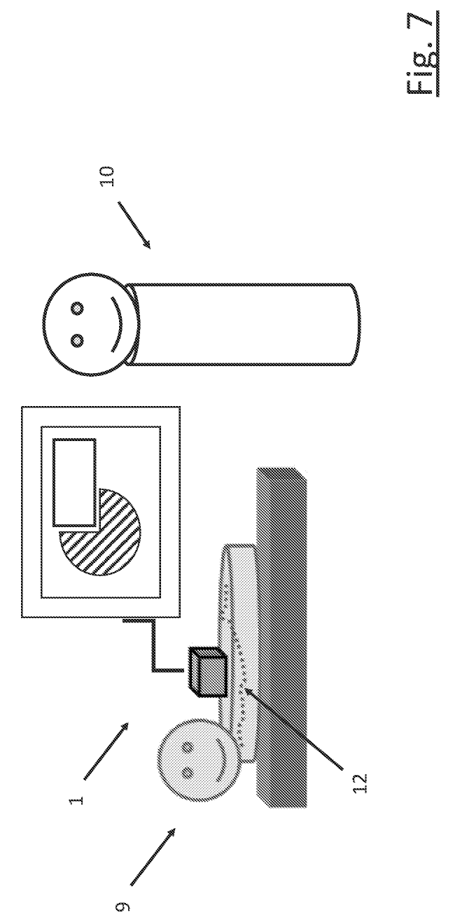

FIG. 7 shows a schematic depiction of an additional user scenario of a mobile rheometric device 1 according to an embodiment of the present invention. Here this can be, for example, a mobile rheometric device 1 according to FIG. 1.

The mobile rheometric device 1 in this case is depicted in direct contact with a patient 9. It is conceivable here that the mobile rheometric device 1 and/or the related rheological measurement device 2 can be positioned directly inside the body of the patient 9 or only on the body of the patient 9. In other words, the diagnosis can either be performed directly inside or on the body, and thus the mobile rheometric device 1 with its corresponding components is configured for both variants.

Contrary to the depiction in FIG. 5, no opened site of the body is depicted at the abdominal level of the patient 9. The mobile rheometric device 1 and/or the related rheological measurement device 2 is merely positioned on the bodily surface of the patient 9.

Before the actual rheological measurements, a bodily tissue or liquid of the patient 9 is stained with a contrasting agent in this region. Thereafter, by means of tomographic methods or the like, at least one result is obtained, such as from the flow behavior or some viscoelastic behavior. In the flow behavior, for instance, a streaming profile can be obtained. This is portrayed in FIG. 7 on the basis of a schematically roughly sketched blood vessel 12, in which correspondingly stained blood flows.

To this extent it is conceivable that by means of the mobile rheometric device 1 and/or by means of the related rheological measurement device 2, a streaming profile of blood can be ascertained in the blood vessels 12. Viscoelastic modes of behavior of samples can be cited by the mobile rheometric device 1 and/or the related rheological measurement device 2, in that tissue structures, for example, or structures in general of the sample, are set in motion by the build-up of an electromagnetic field, in particular one built up temporarily, and thus a corresponding rheological characteristic can then be ascertained.

A user 10 of the mobile rheometric device 1 is depicted on the right, with reference to the image plane. This can, for instance, be a medical staff person, such as an operator.

In an embodiment that is not depicted in greater detail, it is conceivable that the sample of the patient 9, for example synovial fluid, blood, a pericardial fluid, or cerebrospinal fluid, is extracted from the body for the investigation and subsequently can be directed back into the body. The mobile rheometric device 1 and/or the related rheological measurement device 2 can be specially configured for these purposes.

For example, the mobile rheometric device 1 and/or the related rheological measurement device 2 can include means for receiving an insertable hose with the essentially fluid sample, conducting it onward by the mobile rheometric device 1 and/or the related rheological measurement device 2, and then directing it back by another hose to the body of the patient 9. Instead of a hose, other transport apparatuses can be employed, although a hose, for instance one essentially made of a plastic or the like, is advantageous because it can be flexibly bent within the area for the aforementioned actions, so that a comfortable device 1 results.

It is also conceivable in this context that the extracted fluid and/or the essentially fluid sample is not subsequently returned to the body but instead is disposed of in accordance with prevailing regulations.

1. A mobile rheometric device for medical diagnosis of human, animal, or plant samples comprising a rheological measurement device, to which is connected an evaluation unit having an evaluation program as well as, connected thereto, an output unit, distinguished in that the evaluation unit with evaluation program is configured to classify the measured rheological parameters of a human, animal, or plant sample with respect to at least one medical diagnosis or into relevant parameter groups for a medical diagnosis, so that a detailed view of the sample can be provided to a user of the mobile rheometric device by means of the output unit.

2. The mobile rheometric device according to claim 1, wherein the evaluation unit and evaluation program can be connected, for purposes of classification, with at least one database for the medical diagnosis of human, animal, or plant samples on the basis of rheological parameters, or at least comprise such a database.

3. The mobile rheometric device according to claim 2, wherein the evaluation program can be adjusted by means of at least one user-defined input, so that individualized use of the respective database is possible.

4. The mobile rheometric device according to claim 3, wherein the at least one user-defined input is selected from: gender-sensitive input, nourishment input concerning human or animal subjects, medication intake by a human or animal subject, fertilizer application to plant subjects, plant protection application to plant subjects, at least one data element on correlated influences of medications and pathological modifications in at least one organism, suspected diagnoses, at least one data element from a patient registry from at least one country, at least one data element concerning a prior ailment of the organism that is to be studied.

5. The mobile rheometric device according to claim 1, wherein the rheological measurement device is configured to measure rheological parameters of the human, animal, or plant samples separately or on the organism.

6. The mobile rheometric device according to claim 1, wherein the mobile rheometric device comprises an energy system which is configured to supply the mobile rheometric device with electrical energy autarchically or by means of one external energy supply network.

7. The mobile rheometric device according to claim 1, wherein the mobile rheometric device comprises essentially external materials, particularly stainless steel or titanium, which are appropriate for use in a sterilization procedure, particularly gamma ray sterilization, so that the mobile rheometric device can be utilized after the performance of a sterilization procedure in an operating room or even during production or with a service provider for the sterilization of medication products.

8. The mobile rheometric device according to claim 1, wherein the mobile rheometric device is configured to simultaneously measure and classify a number of samples.

9. The mobile rheometric device according to claim 1, wherein the mobile rheometric device is configured to be controlled by means of at least one external device that can be coupled with the mobile rheometric device.

10. A method for medical diagnosis of human, animal, or plant samples comprising the following steps:
preparation and activation of a mobile rheometric device having a rheological measurement device, to which is connected an evaluation unit having an evaluation program as well as, connected thereto, an output unit, distinguished in that the evaluation unit with evaluation program is configured to classify the measured rheological parameters of a human, animal, or plant sample with respect to at least one medical diagnosis or into relevant parameter groups for a medical diagnosis, so that a detailed view of the sample can be provided to a user of the mobile rheometric device by means of the output unit.;
measurement of a human, animal, or plant sample by means of the mobile rheometric device;
classification of results of rheological measurements with respect to at least one medical diagnosis by means of the mobile rheometric device;
outputting the classified results by means of the mobile rheometric device, so that a detailed view of the sample can be provided to a user of the mobile rheometric device by means of the mobile rheometric device.

11. The method according to claim 10, comprising the following additional steps:
connecting the evaluation unit and its evaluation program of the mobile rheometric device with at least one database for the medical diagnosis of human, animal, or plant samples on the basis of rheological parameters;
adaptation of the evaluation program by means of at least one user-defined input, so that an individualized use of the connected external database is possible.

12. The mobile rheometric device according to claim 2, wherein the rheological measurement device is configured to measure rheological parameters of the human, animal, or plant samples separately or on the organism.

13. The mobile rheometric device according to claim 2, wherein the mobile rheometric device comprises an energy system which is configured to supply the mobile rheometric device with electrical energy autarchically or by means of one external energy supply network.

14. The mobile rheometric device according to claim 2, wherein the mobile rheometric device comprises essentially external materials, particularly stainless steel or titanium, which are appropriate for use in a sterilization procedure, particularly gamma ray sterilization, so that the mobile rheometric device can be utilized after the performance of a sterilization procedure in an operating room or even during production or with a service provider for the sterilization of medication products.

15. The mobile rheometric device according to claim 2, wherein the mobile rheometric device is configured to simultaneously measure and classify a number of samples.

16. The mobile rheometric device according to claim 2, wherein the mobile rheometric device is configured to be controlled by means of at least one external device that can be coupled with the mobile rheometric device.
